SEQUENCE LISTING (3 sequences)

SEQ ID NO: 1 - protein, 9 aa, Artificial Sequence
  Antigen mimotope of high molecular weight melanoma associated antigen (HMW-MAA)
TRLQAVKYP

SEQ ID NO: 2 - protein, 9 aa, Artificial Sequence
  Antigen mimotope of high molecular weight melanoma associated antigen (HMW-MAA)
TRTNPWPAL

SEQ ID NO: 3 - protein, 9 aa, Artificial Sequence
  Antigen mimotope of high molecular weight melanoma associated antigen (HMW-MAA)
TRTQPGRFP

(19) United States
(12) Patent Application Publication
(10) Pub. No.: US 2008/0019992 A1
(43) Pub. Date: Jan. 24, 2008

(54) Antigen mimotopes and vaccine against cancerous diseases

(76) Inventors: Christoph Zielinski, Vienna (AT); Otto Scheiner, Perchtoldsdorf (AT); Erika Jensen-Jarolim, Vienna (AT); Heimo Breiteneder, Vienna (AT); Hubert Pehamberger, Vienna (AT)

Correspondence Address: MILBANK, TWEED, HADLEY & MCCLOY LLP, INTERNATIONAL SQUARE BUILDING, 1850 K STRET, N.W., SUITE 1100, WASHINGTON, DC, 20006, US

(21) Appl. No.: 11/710,472

(22) Filed: Feb. 26, 2007

Related U.S. Application Data
(62) Division of application No. 10/488,424, filed on Nov. 9, 2004, now abandoned.

(51) Int. Cl. A61K 39/00; A61P 35/00; C07K 7/06; C07K 7/08

(52) U.S. Cl. 424/185.1; 530/327; 530/328; 530/329

(57) ABSTRACT

The present invention relates to a vaccine against cancerous diseases and antigen mimotopes associated with the high molecular weight melanoma associated antigen (HMW-MAA). The vaccines and antigen mimotopes are recognized immunologically by the monoclonal HMW-MAA antibody 225.28S and comprise at least one peptide with a length of 6 to 14 amino acids. The inventive vaccine permits active immunization against cancerous diseases associated with the high molecular weight melanoma associated antigen (HMW-MAA), whereby both prophylactic and therapeutic effects can be achieved. Further, the antigen mimotopes can serve to detect the immune response obtained.

The present invention relates to a vaccine against cancerous diseases and antigen mimotopes associated with the high molecular weight melanoma associated antigen (HMW-MAA).

In recent years there has been a steady world-wide increase in melanoma cases. Melanoma is the tumor with the highest increase rate, which is stated as 5% a year in Central Europe and the U.S.A. The annual incidence is currently about 12-15 per 100,000 inhabitants in Central Europe and has doubled within the past 10 years, whereby there has also been a world-wide increase in cases. The world-wide highest incidences are reported from Australia and the southern states of the U.S.A. with about 30 cases per 100,000 inhabitants a year. Melanoma occurs in all age-groups, being a rare event before the 15th year of life. The average age at manifestation is 56 years. Melanoma is by definition a malignant tumor of melanocytes. Malignant proliferation manifests itself in a horizontal growth phase or in a vertical growth phase with nodulation.

An important role in tumor growth and cell adhesion is played by the high molecular weight melanoma associated antigen (HMW-MAA) expressed on over 90% of melanomas or the melanoma associated chondroitin sulfate proteoglycan (MCSP). HMW-MAA consists of a large extracellular domain with 2222 amino acids, a smaller transmembrane segment with 25 amino acids and a cytoplasmic domain consisting of 75 amino acids (EMBL accession number X96753). Due to its high expression rate on melanoma cells and due to its high immunogenicity, a number of monoclonal mouse antibodies have been produced that are being used clinically in different ways.

HMW-MAA is an effective target structure for radiodiagnostic questions and its monoclonal anti-idiotypic antibodies are used in specific immunotherapy of melanoma. In this way, humoral anti-HMW-MAA immunity could be induced in approximately 60% of immunized patients with advanced melanoma in one study (Mittelman A., Chen Z. J., Yang H., Wong G. Y., Ferrone S., Proc. Natl. Acad. Sci. USA, 1992 Jan. 15, 89 (2), 466-70). It was further ascertained that this humoral immunity was associated with a statistically significant prolongation of survival rate (Mittelman A., Chen Z. J., Liu C., Wong G. Y., Hirai S., Ferrone S., Clin. Cancer Res. 1995 Jul. 1, 7, 705-13).

One of the abovementioned monoclonal antibodies is the monoclonal HMW-MAA antibody 225.28S, which is partly referred to as antibody 225.28 in the literature. This antibody is specific to HMW-MAA and has in comparison with other antibodies a special paratope which binds to an epitope of the extracellular domain of HMW-MAA (Ziai M. R., Imberti L., Nicotra M. R., Badaracco G., Segatto O., Natali P. G., Ferrone S., Cancer Res., 1987 May 1, 47 (9), 2474-80).

Anti-idiotypic antibodies have been developed against HMW-MAA antibody 225.28S. These anti-idiotypic antibodies MELIMMUNE1 and MF11-30 were used for specific immunotherapy in clinical studies and showed not only the induction of a humoral immune response (Mittelman A., Chen Z. J., Kageshita T., Yang H., Yamada M., Baskind P., Goldberg N., Puccio C., Ahmed T., Arlin Z., Ferrone S., J. Clin. Invest. 1990 December, 86 2136-2144) but also an induction of specific CTLs (cytotoxic T lymphocytes) against tumor cells (Pride M. W., Shuey S., Grillo-Lopez A., Braslawsky G., Ross M., Legha S. S., Eton O., Buzaid A., Ioannides C., Murray J. L., Clin. Cancer Res. 1998 October, 4 (10), 2363-70).

Although the immune response obtained against anti-idiotypic antibodies is a desirable result, the clinical problems involved, such as the induction of human antibodies against mouse immunoglobulin G (HAMA), cannot be left out of consideration.

For this reason there have been attempts to replace the anti-idiotypic antibodies by mimotopes. For example, Geiser et al (Geiser M., Schultz D., Le Cardinal A., Voshol H., Garcia-Echeverria C., Cancer Res., 1999, Feb. 15, 59 (4), 905-10) identified the antigen epitope of the human melanoma-associated chondroitin sulfate proteoglycan by means of a peptide phage library. Further, Ferrone et al (Ferrone S., Wang X., Recent Results Cancer Res., 2001, 158, 231-5) published a 15-mer peptide obtained from a phage peptide library by means of the 225.28S antibody and having a certain homology with the extracellular domain of HMW-MAA.

It is accordingly the problem of the present invention to provide a vaccine against cancerous diseases or an antigen mimotope that are associated with the high molecular weight melanoma associated antigen (HMW-MAA) which make it possible to avoid the disadvantages of conventional cancer treatments, permit effective prophylaxis of such cancerous diseases, and provide an agent for treating such cancerous diseases.

The invention is based on the finding that such a vaccine can be obtained if it contains antigen mimotopes associated with HMW-MAA or their functional variants as effective components.

The subject matter of the present invention is therefore firstly a vaccine against cancerous diseases associated with the high molecular weight melanoma associated antigen (HMW-MAA) which is characterized in that it is recognized immunologically by the monoclonal HMW-MAA antibody 225.28S and comprises at least one peptide with a length of 6-14 amino acids and/or a functional nucleic acid sequence for producing said peptide. A functional nucleic acid sequence for producing said peptide refers to any nucleic acid sequence, DNA or RNA, that is able to code for the corresponding peptide. These DNA or RNA molecules can also be present in viral vectors.

The length of the peptide depends on the length of the peptides used for selection, which are sequences with a length of 6 to 14 amino acids. Said amino acid sequences are no longer than 14 amino acids and no shorter than 6 amino acids, not including any immunogenic carriers that do not endanger human health which can be used.

Also not included are non-specific linkers which can be present between peptide sequence and immunogenic carrier and are preferably joined to the peptide sequence or cosynthesized, whether chemically or by genetic engineering, to facilitate coupling to the carrier such as keyhole limpet hemocyanin (KLH), tetanus toxoid (TT), albumen-binding protein (ABP) or bovine serum albumen (BSA) and/or to serve as spacers between peptide sequence and carrier.

The inventive vaccine permits active immunization against cancerous diseases associated with the high molecular weight melanoma associated antigen (HMW-MAA). Thus, a prophylaxis can be obtained against such cancerous diseases, which are usually melanomas. In addition, the inventive vaccine can be used to treat an existing cancerous disease or to accompany conventional cancer treatments. Application of the inventive vaccine can completely or partly avoid the considerable disadvantages of conventional cancer treatments such as chemo- or radiotherapy.

Preferably, the vaccine is phage-free. That is, even if phage-presented peptides with the desired length of 6 to 14 amino acids are used for selecting an effective amino acid sequence with the aid of antibody 225.28S, these phage-presented peptides should not be processed into a vaccine but previously freed from the phage fraction and only then possibly coupled to a carrier employable in particular for humans.

This can be done in the following way. After the single or multiple panning or selection step, one or more peptide-presenting phages are obtained whose corresponding DNA is sequenced, thereby obtaining the DNA sequence equivalent to the mimotope sequence coupled with the corresponding phage DNA sequence, which can in turn be translated into the corresponding amino acid sequence. This amino acid sequence can be produced by way of solid phase synthesis or by genetic engineering. Both the chemical and the genetic engineering methods permit a non-specific linker to be coupled to this mimotope sequence, whereby improved coupling to a desired carrier is achieved, or the linker serves as a spacer between peptide sequence and carrier.

Preferably, the inventive vaccine contains at least one peptide with a length of 8-12 amino acids and/or a functional nucleic acid sequence for producing said peptide.

The length of the peptide depends on the length of the peptides used for selection, which are sequences with a length of 8 to 12 amino acids. Said amino acid sequences are then no longer than 12 amino acids and no shorter than 8 amino acids, not including any immunogenic carriers that do not endanger human health which can be used.

Also not included are non-specific linkers that can be present between peptide sequence and immunogenic carrier and are preferably joined to the peptide sequence or cosynthesized, whether chemically or by genetic engineering, to facilitate coupling to the carrier such as keyhole limpet hemocyanin (KLH), tetanus-toxoid (TT), albumen-binding protein (ABP) or bovine serum albumen (BSA) and/or to serve as spacers between peptide sequence and carrier.

It is further preferable for the inventive vaccine to contain at least one peptide with a length of 9-11 amino acids and/or a functional nucleic acid sequence for producing said peptide.

The length of the peptide depends on the length of the peptides used for selection, which are sequences with a length of 9 to 11 amino acids. Said amino acid sequences are then no longer than 11 amino acids and no shorter than 9 amino acids, not including any immunogenic carriers that do not endanger human health which can be used.

Also not included are non-specific linkers that can be present between peptide sequence and immunogenic carrier and are preferably joined to the peptide sequence or cosynthesized, whether chemically or by genetic engineering, to facilitate coupling to the carrier such as keyhole limpet hemocyanin (KLH), tetanus toxoid (TT), albumen-binding protein (ABP) or bovine serum albumen (BSA) and/or to serve as spacers between peptide sequence and carrier.

It is especially preferable for the inventive vaccine to contain at least one peptide with an amino acid sequence selected from the following amino acid sequences: TRLQAVKYP, TRTNPWPAL, TRTQPGRFP, TRTKAWPSP, CSLPYIARYAC, CGPRCTGPRCC and CQLPPSAQYAC, and/or a functional peptide variant of these amino acid sequences that can be obtained by substitution, addition and/or omission of one or more amino acids of these amino acid sequences, and/or a functional nucleic acid sequence for producing said amino acid sequences or functional peptide variants. In particular, this refers to peptides having conservative substitution without losing their property as an antigen mimotope. The inventive peptides or their functional variants can also be linked with other peptides or polypeptides or with further chemical groups such as glycosyl groups, lipids, phosphates, acetyl groups or the like, provided they do not adversely influence their effect. Further, these sequences might also be coupled to a non-specific linker that serves as a spacer to the immunogenic carrier or permits improved coupling thereto.

In a preferred embodiment, the peptide or its functional variant is conjugated to an immunogenic carrier. Such carriers can be macromolecules of any kind, it being important that a selected carrier is nontoxic to animals and in particular to humans and involves no dangers e.g. of a phage or phage particle with respect to any contained toxins or the possibility of infection e.g. of intestinal bacteria, and is nonpoisonous and does not trigger any serum sicknesses or food allergies. The vaccine is thus phage-free, that is, even if phage-presented peptides with the desired length of 6 to 14 amino acids are used for selecting an effective amino acid sequence with the aid of antibody 225.28S, these phage-presented peptides should not be processed into a vaccine but previously freed from the phage fraction and only then possibly coupled to a carrier that is employable and completely harmless in particular for humans, and is therefore very suitable for vaccination in the human system. Conjugation to a carrier has the consequence of increasing the immunogenicity of the vaccine.

Examples of carriers that might be stated are keyhole limpet hemocyanin (KLH), tetanus toxoid (TT), albumen-binding protein (ABP) or bovine serum albumen (BSA).

The peptide or its functional variant is preferably conjugated to keyhole limpet hemocyanin (KLH) or tetanus toxoid (TT).

Conjugation of the peptides or their variants to the carrier material can be done in any way, for example by genetic engineering or by chemical means, i.e. carrier and functional group are linked by a chemical reaction. By genetic engineering the protein carrier molecule can be coupled with the peptide or its variant by inserting a DNA or RNA sequence coding for the total sequence of the conjugate into an expression system by which the total conjugate is then expressed. This form of conjugation can of course only be applied if the total conjugate is a protein molecule.

Preferably, the peptides or their variants are conjugated to the carrier by chemical means. That is, the linkage of peptide or its variant and the carrier to the conjugate is effected by chemical means.

The peptides or their functional variants can be conjugated to the carrier as mono-, di-, tri- or oligomer. Such conjugations are described for example in the print by Th. H. Turpen, F. J. Reinel, Y. Charoenvit, S. L. Hoffmann, V. Fallarme in Bio/Technology 1995, Vol. 13, pages 53-57, by the example of conjugation of epitopes to macromolecular carriers. The disclosure of this print is incorporated herein by reference. The described procedures can be applied analogously to the production of the conjugates for the inventive vaccine.

If the conjugation of a di- or oligomeric peptide conjugate is performed using the above-described genetic engineering method, the DNA or RNA portions coding for the peptides are integrated lined up one after the other once or several times into the DNA or RNA sequence coding for the carrier. This obtains the expression of di- or oligomeric peptide conjugates.

The mono- or oligomers of the peptides or their functional variants can be conjugated to the carrier both in single and in multiple form, i.e. one or more peptide molecules or their functional variants are attached to a carrier.

The inventive vaccine can be applied in different ways. The vaccines containing the peptides themselves or their functional peptide or mimotope variants can be administered for example intravenously, subcutaneously or else by oral taking of the vaccine in capsule or tablet form. If the inventive vaccine contains functional nucleic acid variants of the peptides, administration can also be done using an ex-vivo procedure, which comprises removal of cells from an organism, penetration of the inventive vaccine into these cells, and repenetration of the treated cells into the organism.

The inventive vaccine can be produced in diverse ways by genetic engineering or chemical means. If chemical means are used, solid phase peptide synthesis is expedient.

An example of a genetic engineering production method is manipulation of microorganisms such as E. coli. These are manipulated so that they express the peptides as such or the total conjugates consisting of peptide and carrier coupled thereto.

Preferably, the peptides, functional peptide variants or mimetic peptide variants are prepared synthetically by chemical means. In a preferred embodiment, this is done with the aid of solid phase synthesis. It is further preferable for the synthetically produced peptide, the functional peptide variant or mimetic peptide variant to be linked with a carrier such as KLH or TT by chemical means.

The inventive vaccine can be used for prophylactic and acute treatment of humans and animals capable of developing kinds of cancer associated with the high molecular weight melanoma associated antigen (HMW-MAA).

The subject matter of the present invention is further an antigen mimotope of the extracellular domain of the high molecular weight melanoma associated antigen (HMW-MAA) which is characterized in that it is recognized immunologically by the monoclonal HMW-MAA antibody 225.28S and comprises at least one peptide of an amino acid sequence with a length of 6-14 amino acids. The length of the peptide depends on the length of the peptides used for selection, which are sequences with a length of 6 to 14 amino acids. Said amino acid sequences are then no longer than 14 amino acids and no shorter than 6 amino acids, not including any immunogenic carriers that do not endanger human health which can be used.

Also not included are non-specific linkers that can be present between peptide sequence and immunogenic carrier and are preferably joined to the peptide sequence or cosynthesized, whether chemically or by genetic engineering, to facilitate coupling to the carrier such as keyhole limpet hemocyanin (KLH), tetanus toxoid (TT), albumen-binding protein (ABP) or bovine serum albumen (BSA) and/or to serve as spacers between peptide sequence and carrier.

The inventive antigen mimotope can firstly be an essential component of the above-described vaccine, but it is secondly also suitable for monitoring the obtained immune response in a vaccinated patient. It can thus be applied both as a vaccine component and as a diagnostic means in vitro for monitoring the success of a vaccination.

In a preferred embodiment, the antigen mimotope comprises at least one peptide with a length of 8-12 amino acids. The length of the peptide then depends on the length of the peptides used for selection, which are sequences with a length of 8 to 12 amino acids. Said amino acid sequences are then no longer than 12 amino acids and no shorter than 8 amino acids, not including any immunogenic carriers that do not endanger human health which can be used.

Also not included are non-specific linkers that can be present between peptide sequence and immunogenic carrier and are preferably joined to the peptide sequence or cosynthesized, whether chemically or by genetic engineering, to facilitate coupling to the carrier such as keyhole limpet hemocyanin (KLH), tetanus toxoid (TT), albumen-binding protein (ABP) or bovine serum albumen (BSA) and/or to serve as spacers between peptide sequence and carrier.

In a further preferred embodiment, the antigen mimotope comprises at least one peptide with a length of 9-11 amino acids. The length of the peptide depends on the length of the peptides used for selection, which are sequences with a length of 9 to 11 amino acids. Said amino acid sequences are then no longer than 11 amino acids and no shorter than 9 amino acids, not including any immunogenic carriers that do not endanger human health which can be used.

Also not included are non-specific linkers that can be present between peptide sequence and immunogenic carrier and are preferably joined to the peptide sequence or cosynthesized, whether chemically or by genetic engineering, to facilitate coupling to the carrier such as keyhole limpet hemocyanin (KLH), tetanus toxoid (TT), albumen-binding, protein (ABP) or bovine serum albumen (BSA) and/or to serve as spacers between peptide sequence and carrier.

In an especially preferred embodiment, the antigen mimotope comprises at least one peptide with an amino acid sequence selected from the following sequences: TRLQAVKYP, TRTNPWPAL, TRTQPGRFP, TRTKAWPSP, CSLPYIARYAC, CGPRCTGPRCC and CQLPPSAQYAC, and/or a functional peptide variant of these sequences that can be obtained by substitution, addition and/or omission of one or more amino acids of these sequences.

To find the amino acid sequences for the vaccine or the antigen mimotope, a method is applied by which phage libraries presenting peptides with a certain sequence length are bound to HMW-MAA antibody 225.28S in different strength. The phage libraries represent a great variety of sequence compositions with a certain peptide length and are selected in this panning so that only those peptide sequences are selected that have the highest affinity to the antibody. After several repetitions of this process with the particular selected peptides it is possible to isolate such sequences with the highest affinity to the antibody. Identification of the corresponding amino acid sequence is done by conventional genetic engineering methods. The found sequences need not necessarily have a sequence homology to the extracellular domain of HMW-MAA. It suffices it they are able to bind to the paratope of HMW-MAA antibody 225.28S due to their structural properties.

Alternatively to the method using phage libraries, one can also use chemically produced peptide libraries which have been obtained for instance by combinational chemistry e.g. on the solid phase.

The specifically stated peptide sequences can thus vary, provided individual substitutions, additions and/or omissions of one or more amino acids do not strongly impair the function of the peptide, i.e. its ability to bind to the paratope of antibody 225.28S. The inventive peptides or their functional peptide variants can also be linked with other peptides or polypeptides or with further chemical groups such as glycosyl groups, lipids, phosphates, acetyl groups or the like, provided they do not adversely influence their effect.

Preferably, the antigen mimotope is phage-free. That is, even if phage-presented peptides with the desired length of 6 to 14 amino acids are used for selecting an effective amino acid sequence with the aid of antibody 225.28S, these phage-presented peptides should not be processed into a vaccine but previously freed from the phage fraction and only then possibly coupled to a carrier that is employable and completely harmless in particular for humans.

Further, these sequences might also be coupled to a non-specific linker which serves as a spacer to the immunogenic carrier or permits improved coupling thereto.

Preferably, the antigen mimotope, i.e. the peptide or its functional variant, possibly together with linker, is conjugated to an immunogenic carrier. It is further preferable for keyhole limpet hemocyanin (KLH) or tetanus toxoid (TT) to be used as a carrier. However, other carriers can also be used, such as bovine serum albumen (BSA) or albumen-binding protein (ABP). The carrier should be completely harmless for animals and in particular for humans, i.e. be nontoxic or not trigger any serum sicknesses or food allergies for instance.

If the antigen mimotope is used as a diagnostic means, it is preferably conjugated to an immunogenic carrier that was not used for the previous vaccination. When monitoring the success of vaccination, this prevents the diagnostic means from reacting to antibodies that were formed against the carrier fraction of the vaccine and therefore do not serve the purpose of prophylaxis or therapy.

Like the inventive vaccine, the antigen mimotopes can be produced both by chemical means and by genetic engineering. Further, it is possible to couple the antigen mimotope to the carrier as a monomer, dimer, trimer, etc. Furthermore, the antigen mimotope can be bound to the carrier singly or multiply.

Hereinafter the inventive method will be described in detail.

Antibody 225.28S (commercially purchased from Nycomed Amersham Sorin S.R.L., published in Wilson B. S., International J. of Cancer 1981, 28, 293-300) is used in the inventive method to select from phage peptide libraries suitable peptide mimotopes of HMW-MAA against which the antibody is specifically effective. An overview of phage peptide libraries and corresponding literature is given by M. B. Zwick, J. Shen and, J. K. Scott in Current Opinion in Biotechnology 1998, 9: 427-436. The disclosure of this print is incorporated herein by reference.

Phage peptide libraries consist of filamentous phages that express different peptides on their surface in a very great variation range. By conventional selection methods the suitable peptide mimotopes are found using the antibodies effective against the special antigen from these libraries. It should be noted that the found mimotopes do not have to match the corresponding epitope of the antigen in their chemical nature.

The mimotopes selected in this way are characterized by DNA sequencing of the phage DNA. According to the pattern of the found sequences designating the mimotope sequences, mimotopes are produced as fusion protein with a macromolecular carrier or synthesized chemically and conjugated to the macromolecular carrier chemically. This conjugation can be done for example by connecting keyhole limpet hemocyanin with the mimotope protein.

The step of conjugating the mimotopes to a macromolecular carrier not displaying a phage or phage particle guarantees that an immune response of the body is induced upon administration of the vaccine, i.e. this step is done to make the mimotopes more strongly immunogenic.

The expression or production of the found mimotope proteins can also be done by conventional methods, for example expression in E. Coli bacteria.

Hereinafter the present invention will be illustrated further by an embodiment.

### EXAMPLE

Selection of Specific Phages From a pVIII-9aa or a pVIII-11aa Phage Peptide Library (Performed by Schafer-N, Denmark)

For each selection round, polystyrene vessels (Nunc, Naperville, Ill.) were coated overnight at 4° C. with 10 μl of monoclonal mouse antibody 225.28S per ml of coating buffer (50 mM NaHCO₃, pH 9.4). The vessels were then saturated with saturation buffer (5 mg per ml dialyzed bovine serum albumen (BSA) and 0.02% NaN₃ in 0.1 M NaHCO₃, pH 9.6) and washed thoroughly with phosphate-buffered salt solution (PBS), 0.1% Tween 20. Incubation was effected with 10¹² phages of a phage library pVIII-9aa or a phage library pVIII-11aa which were previously left at room temperature for one hour. After washing with PBS/0.1% Tween 20 the bound phages were eluted with glycin-HCL, pH 2.2, and neutralized with 1 M tris-HCl, pH 9.1. The eluted phages were amplified by infecting Escherichia coli TG1 at an OD₆₀₀ of 1.5, and purified with a 20% PEG/2.5 M NaCl precipitation. Two further selection rounds were performed against antibody 225.28S. After 3 selection rounds, screening of the colony was performed (Felici F, Castagnoli L, Musacchio A, Japelli R, Cesareni G. Selection of antibody ligands from a large library of oligopeptides expressed on a multivalent exposition vector. J. Mol. Biol. 1991 Nov. 20, 222(2), 301-310). The phages were purified from single colonies and monitored for their reactivity with 225.28S by phage-enzyme-linked immunosorbent assay (ELISA).

2. Third Round of Phage Pool Analysis

The pool of phages from the third selection round was analyzed by sequencing single phage clones. The phage DNA was purified from the cultures of single phage clones left overnight. DNA sequencing was done by the Sanger Dideoxy method, using a pVIII-specific fluorescence-labeled primer (Leither A, Vogel M, Radauer C, Breiteneder H, Stadler B M, Scheiner O, Kraft D, Jensen-Jarolim E, A mimotope defined by phage display inhibits IgE binding to the plant panallergen profilin. Eur. J. of Immunol. 1998 September, 28(9), 2921-7) and analyzing by means of a LI-COR DNA sequencer 4000 L (LI-COR, Lincoln, Neb.).

3. Phage ELISA

The wells of a 96 microtiter plate (Maxisorp, Nunc) were coated overnight at 4° C. with 2 μl of anti-pill antibody (Dente L, Cesarini G, Micheli G, Felici F, Folgori A, Luzzago A, Monaci P, Nicosia A, Delmastro P, Monoclonal antibodies that recognise filamentous phage: Tools for phage display technology, Gene. 1994 Oct. 11, 148(1), 7-13) per ml of coating buffer. The plates were saturated for two hours at room temperature (5% skimmed milk and 0.05% Tween 30 in PBS, pH 7.3) and washed (0.05% Tween 20 in PBS, pH 7.3). The purified phages were diluted with saturation buffer and the plates were incubated with the dilutions for 3 hours at room temperature. After further washing and incubation with 1 μg of 225.28S per ml of saturation buffer, bound IgG was detected by using AP-conjugated anti-mouse IgG (Sigma), followed by addition of p-nitrophenylphosphate (Sigma). Absorption was measured at 405 nm.

4. Peptide Synthesis

The peptides TRLQAVKYP, TRTNPWPAL, TRTQPGRFP, TRTKAWPSP, CSLPYIARYAC, CGPRCTGPRCC and CQLPPSAQYAC were synthesized by piCHEM (Graz, Austria). The purity of the peptides was higher than 95%, determined by HPLC (high-performance liquid chromatography).

1.-15. (canceled)

16. A composition comprising a peptide of 6 to 14 amino acids comprising an amino acid sequence of SEQ ID NO: 2 or 3.

17. The composition of claim 16, wherein the peptide is of 8 to 12 amino acids.

18. The composition of claim 16, wherein the peptide is of 9 to 11 amino acids.

19. The composition of claim 16 comprising the peptide described by the amino acid sequence of SEQ ID NO: 2.

20. The composition of claim 16 comprising the peptide described by the amino acid sequence of SEQ ID NO: 3.

21. The composition of claim 16, wherein the at least one peptide is conjugated to one or more immunogenic carriers.

22. The composition of claim 21, wherein the one or more immunogenic carriers is tetanus toxin (TT) or keyhole limpet hemocyanin.

23. The composition of claim 19, wherein the at least one peptide is conjugated to one or more immunogenic carriers.

24. An antigen mimotope comprising a peptide of 6 to 14 amino acids comprising an amino acid sequence of SEQ ID NO: 2 or 3, wherein said antigen mimotope is recognized immunologically by the monoclonal antibody 225.28S against high molecular weight melanoma associated antigen (HMW-MAA).

25. The antigen mimotope of claim 24 comprising a peptide of 8 to 12 amino acids.

26. The antigen mimotope of claim 24 comprising a peptide of 9 to 11 amino acids.

27. The antigen mimotope of claim 24 comprising the peptide described by the amino acid sequence of SEQ ID NO: 2.

28. The antigen mimotope of claim 24 comprising the peptide described by the amino acid sequence of SEQ ID NO: 3.

29. The antigen mimotope of claim 24, wherein said peptide is conjugated to one or more immunogenic carriers.

30. The antigen mimotope of claim 27, wherein said peptide is conjugated to one or more immunogenic carriers.

31. The antigen mimotope of claim 29, wherein the one or more immunogenic carriers is tetanus toxin (TT) or keyhole limpet hemocyanin.

32. The antigen mimotope of claim 30, wherein the one or more immunogenic carriers is tetanus toxin (TT) or keyhole limpet hemocyanin.

33. The composition of claim 23, wherein the one or more immunogenic carriers is tetanus toxin (TT) or keyhole limpet hemocyanin.
